(19) United States
(12) Patent Application Publication
(10) Pub. No.: US 2020/0087612 A1
(43) Pub. Date: Mar. 19, 2020

(54) DEVICE FOR DERIVING ANTARCTIC CHIONODRACO RASTROSPINOSUS SKIN ANTIFREEZE PROTEIN

(71) Applicant: Liang Chih Yang, Taipei City (TW)

(72) Inventor: Liang Chih Yang, Taipei City (TW)

(21) Appl. No.: 16/134,987

(22) Filed: Sep. 19, 2018

(51) Int. Cl. C12M 1/33; B06B 3/00; B01J 19/10; C12M 1/00; C12M 3/08; C12M 3/06

(52) U.S. Cl. CPC C12M 45/02; B06B 3/00; B01J 19/10; C12M 3/06; C12M 45/09; C12M 3/08; C12M 45/04

(57) ABSTRACT

A device for manufacturing Antarctic chionodraco rastrospinosus skin antifreeze protein includes a de-frozen and cleaning device for de-frosting frozen grass carp skin and then cleaning carp skin; a skin slurry for making de-fat skin slurry; upper fat on the skin slurry being removed to get de-fat skin slurry; an ultrasonic generator for ultrasonic treatment to change organizational structure of skin antifreeze proteins of the slurry; a composite protease adder for adding composite protease added to the slurry; a collector receiving the slurry from the adder, and then the slurry are retained a time period and cooled to room temperature; then a 4000 g centrifugation is performed through 10 min; and a two-step filter connected to the collector for receiving the supernatant from the collector for performing a two-step ultra-filtration process so as to get skin antifreeze protein with molecular weight of 8000˜10000 Dalton and.

### FIELD OF THE INVENTION

The invention relates to aquatic food processing, in particularly to a device for deriving antarctic chionodraco rastrospinosus skin antifreeze protein.

### FIELD OF THE INVENTION

The invention relates to food processing devices, and in particular to device for manufacturing skin antifreeze protein.

### BACKGROUND OF THE INVENTION

Antifreeze proteins can be bonded to specific surfaces of ice crystals, while does not reduce the freezing point of the solution or inhibit ice crystal growth. Antifreeze proteins can be used in human or animal organ cryopreservation, and improve the quality of its freezing. Frost can improve the ability of certain microorganisms, decreases food frozen ice crystal formation and recrystallization destruction of food structure so as to improve quality of foods. Antifreeze proteins can be used as an advanced antifreeze applications in medicine, cosmetics, biological and food fields and many other applications.

Currently, antifreeze proteins and polypeptides prepared skin resources does not be reported in any document. Therefore there is an eager demand for making skin preparation antifreeze proteins with a simple and efficient way to get the antifreeze protein specific antifreeze function.

### SUMMARY OF THE INVENTION

In order to solve the above problems, an object of the invention is to provide a device for manufacturing skin antifreeze protein. In summary, the invention is directed to product development device for skin antifreeze protein. After a lot of experiments, developed a simple and efficient device for producing skin antifreeze protein. The obtained skin antifreeze protein has a specific function which is better than anyone of the prior art, and is easier to achieve the object of industrial mass production.

### BRIEF DESCRIPTION OF THE DRAWING

FIG. 1 is a block diagram about the structure of the invention.

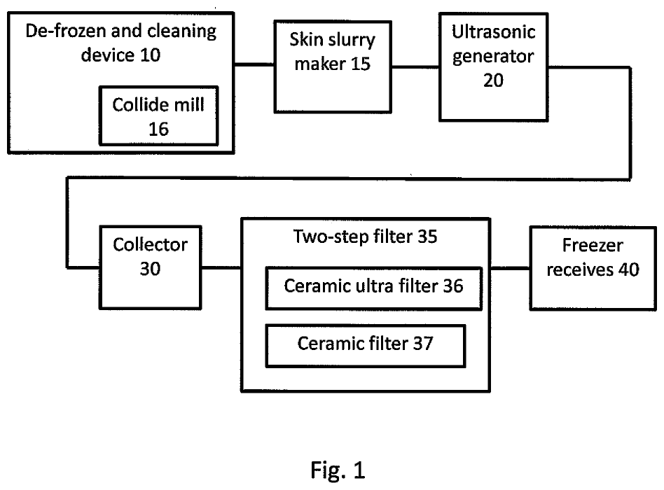

### DETAILED DESCRIPTION OF THE INVENTION

In order that those skilled in the art can further understand the invention, a description will be provided in the following in details. However, these descriptions and the appended drawings are only used to cause those skilled in the art to understand the objects, features, and characteristics of the invention, but not to be used to confine the scope and spirit of the invention defined in the appended claims.

In the invention, the molecular weight of the Antarctic chionodraco rastrospinosus skin antifreeze proteins in the supernatant is 2600˜33000 Dalton; and molecular weights of skin antifreeze protein in liquid skin and skin antifreeze protein products are between 8000˜10000 Dalton. Their ratios in the corresponding products (supernatant, skin and skin protein liquid antifreeze protein products) are more than 95% under consideration of protein molecular.

The device of the invention is operated under room temperature of 25 degree C.; the pH value is got under the environment of 1 mol/L of NaOH and HCl. The protein content of the skin antifreeze protein product is determined by Kjeldahl method (GB 5009.5-2010). The molecular weight of the skin antifreeze proteins in the supernatant is between 2600˜33000 Dalton; and molecular weights of protein liquid skin and skin antifreeze protein products are 8000˜10000 Dalton; which is more than 95 weight percentage of the corresponding products (supernatant, skin and skin protein liquid antifreeze protein products).

The invention contains the following elements.

A de-frozen and cleaning device 10 serves for defrosting 100 grams of frozen Antarctic chionodraco rastrospinosus skin through 12 hours at 7° C. The Antarctic chionodraco rastrospinosus skin is cleaned with cleaning water at a temperature of 10° C.

A skin slurry maker 15 is connected to the de-frozen cleaning device 10 for making de-fat skin slurry; in that after the skin is cleaned in the de-frozen cleaning device 10, the skin is beat by a beater (not shown) into skin pulp; then water with weight of 1.5 times of the skin pulp is added thereto. A colloid mill 16 grinds the skin pulp into skin slurry; then by 6000 g-centrifuging operation to the skin slurry through 8 mins, upper fat on the skin slurry is removed to get de-fat skin slurry of 245 grams.

An ultrasonic generator 20 is connected to the skin slurry maker 15. It serves to receive the de-fat skin slurry; and then by ultrasonic treatment with a power of 800 w and frequency of 25 KHz through 10 minutes to change organizational structure of skin antifreeze proteins of the slurry.

A composite protease adder 25 is connected to the ultrasonic generator 20, wherein a 0.2 grams of composite protease is added to the slurry processed by the ultrasonic generator. The composite protease is a composition of alkaline protease and neutral protease which are formed by a mass ratio of 1:1. Under a condition of 50° C., and a pH value of 7.5, the slurry was digested through 0.5 hour.

A collector 30 is connected to the composite protease adder 25 and serves to receive the slurry from the composite protease adder 25, and then the slurry are retained at a temperature of 95° C. through 3 minutes and next it is cooled to room temperature. Then a 4000 g centrifugation is performed through 10 min. Then supernatant at an upper layer of the slurry is collected, which has a weight of 143 g. Molecular weight of skin antifreeze protein in the supernatant is between 2600˜33000 Daltons.

A two-step filter 35 is connected to the collector 30 serve to receive the supernatant from the collector for performing a two-step ultra-filtration process. In a first step, a ceramic ultra-filter 36 with pore size of 0.5 um is used to filer the supernatant. After filtering, proteins with the molecular weight less than 10000 is separated from polypeptides. Then a ceramic filter 37 with pore size of 2 um is further used, and then the proteins with molecular weight of 8000˜40000 Dalton is separated so as to get 85 grams of skin antifreeze protein with molecular weight of 8000˜10000 Dalton.

A freezer 40 receives the 8000˜40000 Dalton skin antifreeze protein solution in above step; and then concentrate and freeze-dry the skin antifreeze protein to obtain a 5.2 gram of skin antifreeze protein with molecular weight of 8000˜10000 Dalton. In that, skin antifreeze protein has a content of 93.8% (by weight).

The invention utilizes ultrasonic technology to deal with skin protein pulp. Mechanical mechanism of action is due to the high radiation pressure and ultrasonic effects of high pressure on the media to generate ultrasonic vibration and shock. The device of the invention can significantly improve the structure of skin antifreeze proteins and thus to improve the yield of protein antifreeze ability and skin products. The invention develops skin antifreeze protein product which is safe, has food-grade composite proteases, and is especially combinations of neutral and alkaline protease under mild conditions and moderate enzyme that is obtained through a specific molecular weight skin antifreeze protein, without any additives, and thus is 100% skin antifreeze proteins.

The invention develops antifreeze protein. It can be widely used to prompt cryogenic protective effect of microorganisms, improving the organizational structure of the ice cream.

In summary, the invention is directed to product development status of the antifreeze protein present, skin as raw materials, after a lot of experiments, developed a simple and efficient method for producing skin antifreeze protein. The obtained skin antifreeze protein has a specific function which is better and easier mass production industrially so that has a high market value.

Although the above has been described by the general and the specific embodiments of the invention has been described in detail, but on the basis of the invention, it may make some changes or improvements, which the skilled artisan apparent. Thus, the invention without departing from the spirit on the basis of these modifications or improvements made, belong to the scope of the invention as claimed.

1. A device for manufacturing Antarctic chionodraco rastrospinosus skin antifreeze protein, comprising:
a de-frozen and cleaning device for de-frosting frozen Esox-luciu carp skin through 12 hours at 7° C.; and then cleaning carp skin with cleaning water under a temperature of approximate 10° C.;
a skin slurry maker connected to the de-frozen cleaning device for making de-fat skin slurry; in that after skin cleaning in the de-frozen cleaning device, the skin is beat by a beater into skin pulp; then water is added thereto; a colloid mill grinds the skin pulp into skin slurry; then by centrifuging operation to the skin slurry, upper fat on the skin slurry being removed to get de-fat skin slurry;
an ultrasonic generator connected to the skin slurry maker which receives the de-fat skin slurry; and then by ultrasonic treatment with a power of 800 w and frequency of 25 KHz through 10 minutes to change organizational structure of skin antifreeze proteins of the slurry;
a composite protease adder connected to the ultrasonic generator, wherein composite protease is added to the slurry processed by the ultrasonic generator; the composite protease is a composition of alkaline protease and neutral protease which are formed by a mass ratio of 1:1; under a condition of 50° C., and pH value of 7.5, the slurry was digested through 0.5 hour;
a collector connected to the composite protease adder which serves to receive the slurry from the adder, and then the slurry are retained at a temperature of 95° C. and next it is cooled to room temperature; then a 4000 g centrifugation is performed through 10 min; then supernatant at an upper layer of the slurry is collected, molecular weight of skin antifreeze protein in the supernatant is between 2600˜33000 Daltons;
a two-step filter connected to the collector for receiving the supernatant from the collector for performing a two-step ultra-filtration process, in a first step, a ceramic ultra filter with pore size of 0.5 um is used to filer the supernatant; after filtering, proteins with the molecular weight less than 10000 is separated from polypeptides; then using a ceramic filter with a pore size of 2 um is further used, and then the proteins with molecular weight of 8000˜40000 Dalton is separated so as to get 85 grams of skin antifreeze protein with molecular weight of 8000˜10000 Dalton and.

2. The device for manufacturing Antarctic chionodraco rastrospinosus skin antifreeze protein of claim 1, further comprising:
a freezer for receiving the 8000˜10000 Dalton skin antifreeze protein solution and then concentrate and freeze-dry the solution to obtain a molecular weight of 8000˜10000 Dalton skin antifreeze protein.
